(19) United States
(12) Patent Application Publication
(10) Pub. No.: US 2026/0337240 A1
(43) Pub. Date: Oct. 29, 2026

(54) CC16-DERIVED ANALOGS FOR THE TREATMENT OF INFLAMMATORY AND OBSTRUCTIVE LUNG DISEASES

(71) Applicant: ARIZONA BOARD OF REGENTS ON BEHALF OF THE UNIVERSITY OF ARIZONA, Tucson, AZ (US)

(72) Inventors: Julie Ledford, Tucson, AZ (US); Josef Vagner, Tucson, AZ (US); Stefano Guerra, Tucson, AZ (US); Michael D. L. Johnson, Tucson, AZ (US)

(21) Appl. No.: 19/711,739

(22) Filed: Jun. 17, 2026

Related U.S. Application Data
(63) Continuation-in-part of application No. PCT/US24/60845, filed on Dec. 18, 2024.
(60) Provisional application No. 63/611,467, filed on Dec. 18, 2023.

(51) Int. Cl. C07K 14/47; A61K 38/00; A61P 11/06

(52) U.S. Cl. CPC C07K 14/4702; A61P 11/06; A61K 38/00

(57) ABSTRACT

A composition for treating inflammatory, neoplastic and obstructive lung diseases featuring a CC16-derived analog is described herein. The analog may include N-terminal acetylation and C-terminal amidation, enhancing stability. Adopting a helical conformation, the CC16-derived analog maintains activity akin to full-length CC16 protein. Effective for diverse lung diseases, the composition allows aerosolization, subcutaneous injection, and pulmonary delivery, with administration options including nasal inhalation, subcutaneous, or oral routes. A method for treating these diseases involves administering the CC16-derived analog to a subject in need. This innovation offers a versatile, efficacious solution for respiratory conditions, providing tailored treatment approaches.

### CROSS-REFERENCE TO RELATED APPLICATIONS

This application is a continuation-in-part and claims benefit of International Application No. PCT/US2024/060845, filed Dec. 18, 2024, which claims priority to U.S. Provisional Application No. 63/611,467, filed Dec. 18, 2023, the entire contents of each are incorporated herein by reference.

### STATEMENT REGARDING FEDERALLY SPONSORED RESEARCH OR DEVELOPMENT

This invention was made with government support under Grant No. HL142769 and AI135108 awarded by National Institutes of Health. The government has certain rights in the invention.

### REFERENCE TO AN ELECTRONIC SEQUENCE LISTING

The contents of the electronic sequence listing (ARIZ 23_35_PCT_CIP.xml; Size: 58,796 bytes; and Date of Creation: Jun. 16, 2026) is herein incorporated by reference in its entirety.

### FIELD OF THE INVENTION

The present invention features CC16-derived analogs for the treatment of inflammatory, neoplastic, and obstructive lung disease.

### BACKGROUND OF THE INVENTION

CC16 (club cell secretory protein, also known as CCSP, CC10, and uteroglobin) is a homodimeric pneumoprotein encoded by the SCGB1A1 gene. Predominantly produced by club cells and nonciliated epithelial cells in the distal airways, CC16 diffuses into the bloodstream and is readily detectable in serum. Extensive research has explored its potential as a biological marker for lung epithelial cell injury. Studies have demonstrated decreased serum CC16 concentrations in patients with obstructive lung diseases, such as asthma and chronic obstructive pulmonary disease (COPD), as well as in individuals with lung function deficits in the general population. In several epidemiological cohorts, low serum CC16 levels have been shown to predict subsequent impaired lung function growth during childhood, as well as accelerated lung function decline and the onset of airflow limitation, a hallmark of COPD, in adulthood. Low serum CC16 levels also predict mortality by lung cancer.

Additional treatments for inflammatory, neoplastic and obstructive lung diseases are urgently needed to restore CC16 levels in patients effectively. The present invention addresses this need.

### BRIEF SUMMARY OF THE INVENTION

It is an objective of the present invention to provide compositions and methods that allow for the treatment of inflammatory, neoplastic and obstructive lung diseases or the common cold (e.g., a rhinovirus), as specified in the independent claims. Embodiments of the invention are given in the dependent claims. Embodiments of the present invention can be freely combined with each other if they are not mutually exclusive.

In some embodiments, the present invention features a composition comprising a CC16-derived analog. In some embodiments, the CC16-derived analog comprises compound C836 according to the sequence DQD-Nle-REAGAQLKKLVDT (SEQ ID NO: 41). Therefore, in other embodiments, the present invention may feature a composition comprising a CC16-derived analog according to the sequence: DQD-Nle-REAGAQLKKLVDT (SEQ ID NO: 41). In some embodiments, the CC16-derived analog comprises a sequence that is at least 100%, 95%, 90%, or 85% identical to a sequence according to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, the present invention features a CC16-derived analog having a structure according to Formula I, or a pharmaceutical composition comprising the CC16-derived analog having a structure according to Formula I.

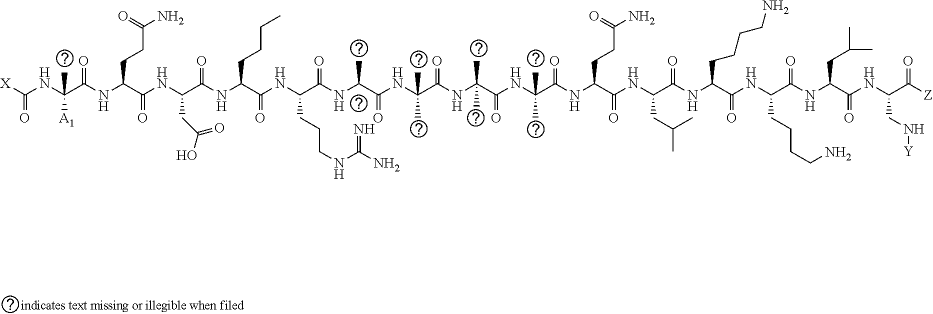

### (Formula I)

wherein X is an alkyl, a branched alkyl, an acetyl, an isopropyl, or an isobutyl; Z is OH, NH₂, NH-alkyl, dipeptide, or an amino acid residue; A is an H, methyl, cyclohexyl, isopropyl, isobutyl, butyl, or an amino acid residue selected from the amino acid group consisting of glutamic acid, aspartic acid, alanine, cyclohexyl glycine, cyclohexyl alanine, amino-isobutyric acid and Y is

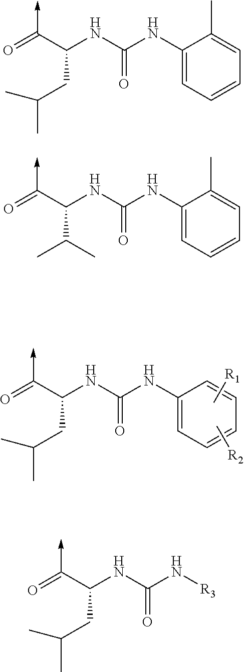

wherein R1, R2 is an H, methyl, a halogen, or OMe, and wherein R3 is a linear or branched alkyl.

In some embodiments, Z is an amino acid residue selected from the group consisting of proline, leucine, valine, and aspartic acid. In some embodiments, Z is a mimic of carboxylic acid selected from the group consisting of hydroxamic acid, urea, N-alkylated urea, N-phenylurea, 2-methyl-N-phenylurea, substituted phenylsulfonamide. In some embodiments, A is an amino acid residue selected from the group consisting of alanine or D-alanine. In some embodiments, R3 is isobutyl or isovaleryl.

In some embodiments, the present invention features a method of treating inflammatory, neoplastic and obstructive lung diseases in a subject in need thereof. In some embodiments, the method comprises administering an effective amount of a CC16-derived analog, e.g., as described herein, to the patient.

The CC16-derived analogs (e.g., peptides) described herein may further comprise an N-terminal acetylation, a C-terminal amidation, or a combination thereof.

In some embodiments, the CC16-derived analogs described herein maintain activity similar to a full-length CC16 protein. In some embodiments, the composition is in a preparation for aerosolization, subcutaneous injection, or pulmonary delivery. In some embodiments, the composition is administered via nasal inhalation, subcutaneously, or orally.

One of the unique and inventive technical features of the present invention is the use of CC16-derived analogs (e.g., peptidomimetics) for the treatment of inflammatory, neoplastic, and obstructive lung disease. Without wishing to limit the invention to any theory or mechanism, it is believed that the technical feature of the present invention advantageously offers multiple advantages over native CC16. Specifically, the analogs are designed to deliver a higher dose of the active region on a mass-per-gram basis, enhancing their therapeutic efficacy. In addition, the removal of oxidizable amino acids reduces the likelihood of activity loss due to oxidation, thereby improving the stability and extending the half-life of the analogs. Furthermore, their smaller molecular size facilitates more efficient delivery compared to CC16.

These combined features-smaller size, optimized active region, and enhanced stability-represent a significant improvement over CC16 and underscore the novelty and inventiveness of the present invention. None of the presently known prior references or works have the unique, inventive technical feature of the present invention.

Moreover, the prior references teach away from the present invention. For example, current treatments for COPD and asthma primarily rely on inhaled corticosteroids, often combined with bronchodilators. In cases of exacerbations, systemic corticosteroids and antibiotics are commonly used. For asthma, biologics have emerged as a treatment option, but their use is typically limited to specific subgroups of severe patients due to their specific targets, high cost, and the need for injections. COPD treatment options are even more limited, with a majority relying on therapies originally developed for asthma. Severe cases may require lung transplants or long-term oxygen therapy, which significantly restricts patients' independence and quality of life. Corticosteroid use across both conditions is associated with numerous long-term side effects, highlighting the need for more effective and accessible treatments.

Furthermore, the inventive technical feature of the present invention contributed to a surprising result. For example, a protein fragment comprising less than 10% of the full CC16 protein can effectively replicate the functional properties of the native CC16 protein. Notably, some of these analogs are capable of upregulating endogenous CC16 gene and protein expression. Another surprising result of the inventive technical features of the present invention is when CC16-derived analogs are delivered into the bloodstream, they impact functions and host defense in the lung, more specifically by reducing airway hyperresponsiveness and inflammation while promoting the secretion of protective factors, including antimicrobials, antivirals, and antioxidants.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows solid-phase synthesis of compound C836. (a) 10% piperidine in DMF, 2+20 minutes. (b) coupling: (i) Fmoc-Thr (tBu), DIC/HOBt (ii) Fmoc-Thr (tBu), HBTU/collidine, in DMF, 2 hrs. (c) general solid-phase procedure. (d) 2.0M acetic anhydride/Pyridine in DCM, 20 minutes (f) general TFA cleavage protocol: 95% TFA, 3% EDT, 2% TIPS, 2 hr. (g) (i) work-up and HPLC purification.

FIG. 2 shows Solid-phase synthesis of cyclic peptide C966. (a) 10% piperidine in DMF, 2+20 minutes; (b) coupling: (i) Fmoc-Thr (tBu), DIC/HOBt (ii) Fmoc-Thr (tBu), HBTU/collidine, in DMF, 2 hrs; (c) general solid-phase procedure. (d) 2.0M acetic anhydride/Pyridine in DCM, 20 minutes; (e) Pd(0)[(Ph3P)4], dimethylbarbituric acid, DCM, 2×30 min; (f) cyclization HBTU/DIEA, DMF, overnight (g) general TFA cleavage protocol: 95% TFA, 3% EDT, 2% TIPS, 2 hr. (h) work up and HPLC purification.

FIG. 3 shows RP-HPLC profiles of compound C836. HPLC was carried out under the following conditions: C836 analyzed using an Agilent 2600 system with a 3.0×100 mm Agilent Poroshell 120 EC-C18 2.7-micron column eluting with a solvent gradient A: B, where solvent A: 0.1% TFA in water and solvent B: 0.09% TFA in acetonitrile:water (9:1), over 15 min at a flow rate of 0.4 mL/min. Rt=7.419 mins.

FIG. 4 shows that rCC16 given to CC16^{-/-} mice decreases human Rhinovirus (RV) burden. RT-PCR at 48 hrs. * p<0.05 by t-test.

FIGS. 5A and 5B show a loss of CC16, which results in increased infectivity of mouse tracheal epithelial cells (MTECs) grown at an air-liquid interface (ALI). FIG. 5A shows Mycoplasma pneumoniae (Mp) burden and FIG. 5B shows RV burden in WT and CC16^{-/-} MTECS after 48 hrs. Cells were grown at ALI for 2 weeks, after which they were infected with either 1×10⁸ Mp CFU/ml or 1×10⁷RV PFU/ml. The burden was determined by RT-PCR using Taqman primers and shown as fold relative to WT. * p<0.05 and **** p<0.0001 by t-test.

FIG. 6 shows that rCC16 and C836 reduce RV infection in human nasal cells. Cells were grown at ALI for 3 weeks and infected with RV for 4 hours, after which they were washed and allowed to incubate for 48 hours with or without rCC16 or C836. RV was determined by qPCR. Each dot is a technical replicate, ** p<0.01 by ANOVA.

FIGS. 7A and 7B show nasal cells from asthma patients are more susceptible to RV infection (FIG. 7A) and have decreased Scgb1a1 (CC16) gene expression (FIG. 7B) compared to non-asthma participants. Cells were grown at ALI for 3 weeks and infected with RV (1×106 PFU) for 4 hrs, after which they were washed and allowed to incubate for 48 hrs. RV and CC16 were determined by qPCR. Each dot is a technical replicate, **** p<0.0001 by ANOVA.

FIG. 8 shows that C836 inhibits neutrophil recruitment and protects lung function. Left panel, C836, was delivered via IV 2 hrs after each HDM challenge. Cells in the lavage fluid were measured 24 hrs after the last HDM challenge. Right panel, baseline lung function, without methacholine provocation, as assessed in the mice receiving C836 IV, 24 hrs after the last HDM challenge. ** p<0.01, **** p<0.0001 by ANOVA.

FIG. 9 shows C836 reduces AHR in allergic airways. C836 was delivered to the airways 2 hrs after each HDM challenge. AHR to methacholine was assessed 24 hrs after the last HDM challenge. * p<0.05 at the highest dose.

FIG. 10 shows screening of human nasal cells for peptidomimetic activity in reducing RV infection. Top panel, RV infectivity; middle panel, IFN-lamda; bottom panel, TNF-alpha levels. All determined by RT-PCR.

FIG. 11 shows the generic structure of compound 1019.

FIG. 12 shows Solid-phase synthesis of compound 1019. (a) Fmoc-Dap(Aloc)-OH, CSCO3, DIEA, DCM, overnight. (b) general solid-phase procedure. (c) 2.0M acetic anhydride/Pyridine in DCM, 20 minutes. (d) Aloc-deprotection Pd(Ph3P)4, N,N-dimethylbarbituric acid, DCM, 30 minutes. (f) General coupling of Fmoc-Leu. (g) 2-methylphenylisocyanate, DMF, overnight. (h) general TFA cleavage protocol: 95% TFA, 3% EDT, 2% TIPS, 2 hr. (i) work-up and HPLC purification.

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

In some embodiments, the peptides described herein are modified by addition of an amine or acid group to the C-terminal, and acetylation or addition of a histidine (H) to the N-terminal. In other embodiments, the peptides described herein are modified by addition of an acid to the N-terminal. Non-limiting examples of acids that may be used to modify the N-terminal of the peptides described herein may include but are not limited to a hydroxyl group (—OH), carboxyl group/carboxylic acid group (COOH), or a combination thereof. In further embodiments, the peptides described herein are modified with a lipid group and the C-terminal and/or N-terminal.

As used herein, the term “wildtype” refers to a non-mutated version of a gene, allele, genotype, polypeptide, or phenotype, or a fragment of any of these. It may occur in nature or be produced recombinantly. As used herein, the term “variant” refers to a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimetics include chemically modified peptides, peptoids (side chains are appended to the nitrogen atom of the peptide backbone, rather than to the α-carbons), P-peptides (amino group bonded to the β carbon rather than the α carbon), etc. As used herein, “peptide mimetic,” “peptidomimetic,” and “analog” may be used interchangeably.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M)

Naturally occurring residues may be divided into classes based on common side chain properties, for example: polar positive (histidine (H), lysine (K), and arginine (R)); polar negative (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

Subject,” “individual,” “host,” “animal,” and “patient” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

As used herein, the terms “administration” and “administering” refer to the act of giving a drug, prodrug, or other agent, or therapeutic treatment (e.g., SP-A peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

“Treatment,” as used herein, covers any administration or application of a therapeutic for disease in a mammal, including a human, and includes inhibiting the disease, arresting its development, or relieving the disease, for example, by causing regression, or restoring or repairing a lost, missing, or defective function; or stimulating an inefficient process.

A “pharmaceutically acceptable carrier” refers to a non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause an injection site reaction.

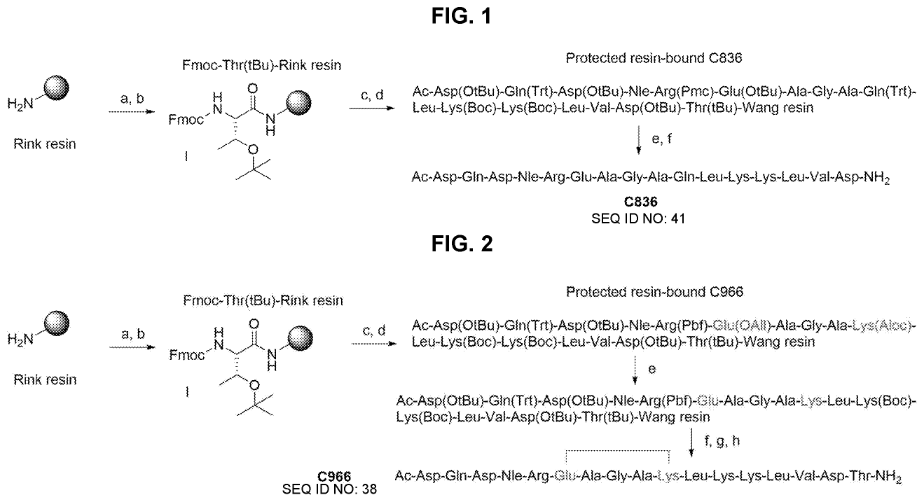

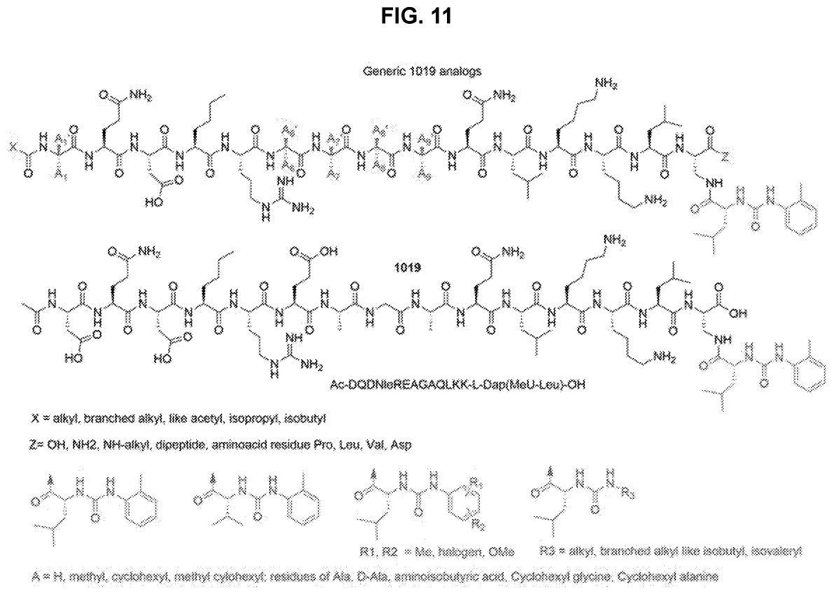

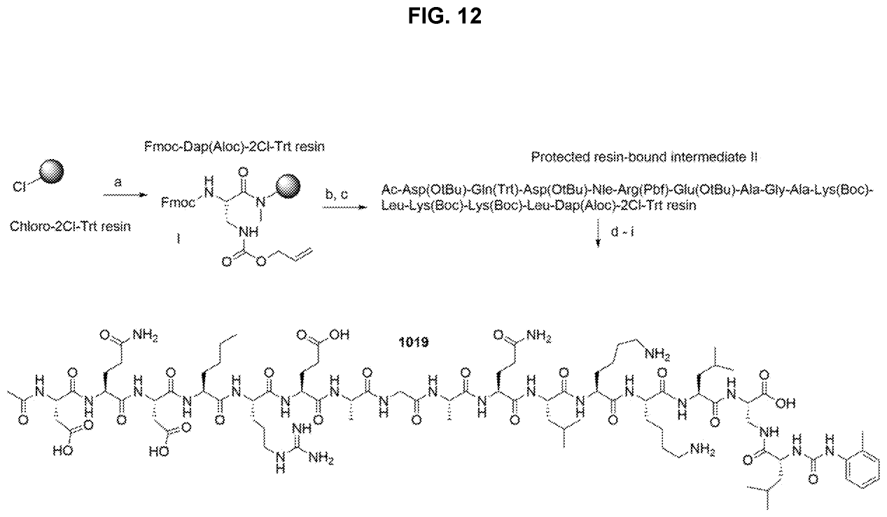

Referring now to FIGS. 1-12, the present invention features CC16-derived peptidomimetics (i.e., analogs) for the treatment of inflammatory, neoplastic, and obstructive lung disease. CC16-derived peptidomimetics offer several advantages over traditional proteins, making them highly attractive for therapeutic development. Their resistance to proteolysis and oxidative degradation improves metabolic stability and extends in vivo half-lives, enhancing pharmacokinetics. Higher stability also improves shelf life, reduces denaturation, and lower degradation during storage. Structural modifications allow for enhanced activity, while precise design can reduce off-target effects. Incorporating non-natural building blocks (amino acids) lowers immunogenicity, as peptidomimetics are less likely to be recognized and targeted by the immune system and are synthesized to minimize biological contamination. Their production relies on straightforward chemical synthesis rather than complex expression systems, offering a cost-effective manufacturing process. Additionally, peptidomimetic modifications can improve membrane permeability, facilitating intracellular delivery, and solubility can be fine-tuned to ensure compatibility with various administration routes through tailored hydrophobicity or hydrophilicity.

In some embodiments, the present invention features a composition comprising a CC16-derived analog. For example, the composition comprises the CC16-derived analog according to the sequence: DQD-Nle-REAGAQLKKLVDT (SEQ ID NO: 41). In some embodiments, the CC16-derived analog comprises a sequence that is at least 100%, 95%, 90%, or 85% identical to a sequence according to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, the present invention features a CC16-derived analog having a structure according to Formula I, or a pharmaceutical composition comprising the CC16-derived analog having a structure according to Formula I.

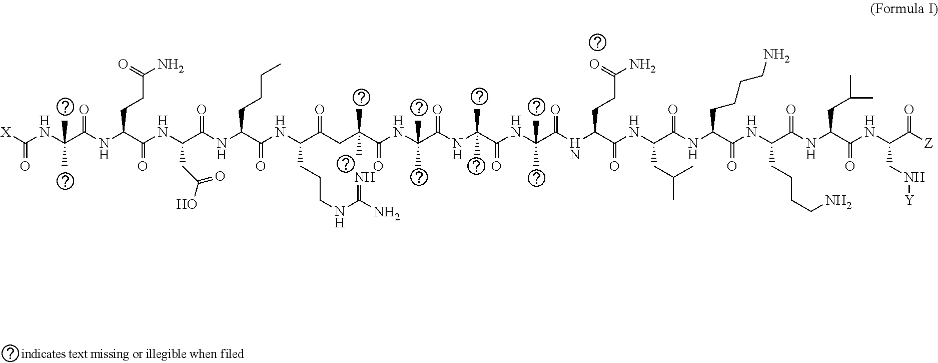

wherein X is an alkyl, a branched alkyl, an acetyl, an isopropyl, or an isobutyl; Z is OH, NH2, NH-alkyl, dipeptide, or an amino acid residue; A is an H, methyl, cyclohexyl, isopropyl, isobutyl, butyl, or an amino acid residue selected from the amino acid group consisting of glutamic acid, aspartic acid, alanine, cyclohexyl glycine, cyclohexyl alanine, amino-isobutyric acid and Y is

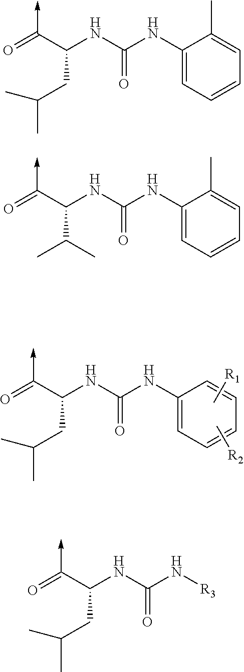

wherein R1, R2 is an H, methyl, a halogen, or OMe, and wherein R3 is a linear or branched alkyl.

In some embodiments, Z is an amino acid residue selected from the group consisting of proline, leucine, valine, and aspartic acid. In some embodiments, Z is a mimic of carboxylic acid selected from the group consisting of hydroxamic acid, urea, N-alkylated urea, N-phenylurea, 2-methyl-N-phenylurea, substituted phenylsulfonamide. In some embodiments, A is an amino acid residue selected from the group consisting of alanine or D-alanine. In some embodiments, R3 is isobutyl or isovaleryl.

Non-limiting examples of the CC16-derived analogs described herein are provided in Table

1.

Table 1:

[symbol]

In some embodiments, the CC16-derived analogs (e.g., peptides) described herein further comprise one or more modifications at the N-terminus, the C-terminus, or both. For example, the CC16-derived analogs may comprise an N-terminal acetylation, a C-terminal amidation, or a combination thereof. Non-limiting N-terminal modifications may include, but are not limited to, acetylation or alkylation (e.g., simple or branched alkylation). Additionally, non-limiting C-terminal modifications may include, but are not limited to, incorporation of aspartic acid residues (e.g., within a dipeptide such as LD, VD, or DG), dipeptide NleG, dG, or a tetrapeptide [symbol] embodiments, the C-terminal modification may comprise an isosteric structure that mimics a C-terminal carboxylate group (—COOH), including, but not limited to, hydroxamic acid (—CONHOH), tetrazole, or sulfonamide (—CONHSO2R).

In some embodiments, the CC16-derived analogs (e.g., peptides) described herein adopt a helical conformation. In other embodiments, the CC16-derived analog comprises one or more modifications that produce a structure that mimics a helical conformation. Without wishing to limit the present invention to any theory or mechanism it is believed that the CC16-derived analogs described herein maintain activity similar to full-length CC16 protein. In some embodiments, the CC16-derived analog is cyclic. In other embodiments, at least a portion of the CC16-derived analog is cyclic.

In some embodiments, the compositions described herein are formulated for pulmonary administration. Pulmonary administration may be achieved by inhalation of an aerosolized formulation, including administration through a metered-dose inhaler, dry powder inhaler, soft mist inhaler, nebulizer, atomizer, inhalation spray device, ventilator circuit, or any other device capable of delivering the composition to the respiratory tract. The aerosolized formulation may comprise droplets, particles, or aerosols having a size suitable for deposition within the upper airways, lower airways, bronchi, bronchioles, alveoli, or combinations thereof.

In some embodiments, the compositions described herein are administered by nebulization. In certain embodiments, the composition is provided as a liquid formulation suitable for aerosolization. In other embodiments, the composition is formulated as a dry powder for inhalation.

In some embodiments, the compositions described herein are administered to a patient receiving assisted ventilation. In certain embodiments, the compositions described herein are administered systemically, including by intravenous administration. In some embodiments, intravenous administration is used in critically ill patients, including patients receiving mechanical ventilation.

In some embodiments, the compositions described herein are useful for the treatment of inflammatory, neoplastic, obstructive, and infectious lung diseases, as well as pulmonary disorders associated with preterm birth. In some embodiments, the composition is in a preparation for aerosolization, subcutaneous injection, or pulmonary delivery. In some embodiments, the composition is administered via nasal inhalation, subcutaneously, or orally.

In other embodiments, the present invention features a method of treating inflammatory, neoplastic, obstructive, and infectious lung diseases, as well as pulmonary disorders associated with preterm birth in a subject in need thereof. The method comprises administering an effective amount of a CC16-derived analog as described herein to the patient. In other embodiments, the method comprises administering an effective amount of a CC16-derived analog according to Formula I or Formula II (compound #1019) to the patient, including, without limitation, any of the CC16-derived analogs set forth in Table 1.

Non-limiting examples of inflammatory, neoplastic, and obstructive lung diseases include but are not limited to asthma, chronic obstructive pulmonary disease (COPD), cystic fibrosis, lung cancer, lung function deficits, and conditions leading to obstructive lung function deficits.

As used herein, “obstructive lung function deficits” refers to conditions that limit airflow, preventing the lungs from effectively taking in oxygen or expelling carbon dioxide. These deficits often result in shortness of breath and reduced lung capacity and are typically diagnosed through pulmonary function tests, with spirometry being a common method. Obstructive lung function deficits are characterized by reduced levels of forced expiratory volume in one second (FEV1) and a reduced ratio between FEV1 and forced vital capacity (FEV1/FVC). An example of a condition leading to obstructive lung function deficits includes preterm birth, which is associated with lower blood levels of CC16, which may contribute to airflow limitation later in life.

Obstructive lung function deficits can have genetic and environmental causes. Some individuals have reduced lung function from birth, while others, who initially had normal lung function, may experience a decline due to events like smoking or pneumonia. In some embodiments, asthma and COPD are both obstructive lung diseases.

The present invention may further feature a CC16-derived analog composition for use in a method of treating inflammatory, neoplastic and obstructive lung diseases in a subject in need thereof. In other embodiments, the present invention may feature a CC16-derived analog composition for use in a method of treating inflammatory, neoplastic and obstructive lung diseases in a subject in need thereof; wherein the CC16-derived analog composition comprises a sequence according to SEQ ID NO: 41 or a derivative thereof.

### Example 1

The following is a non-limiting example of the present invention. It is to be understood that said example is not intended to limit the present invention in any way. Equivalents or substitutes are within the scope of the present invention.

### Non-Standard Abbreviations

As used herein, the following abbreviations may be used interchangeably with their corresponding terms: Aloc refers to allyloxycarbonyl; All refers to allyl; Boc refers to tert-butyloxycarbonyl; DCM refers to dichloromethane; DIC refers to N,N′-diisopropylcarbodiimide; DIEA refers to diisopropylethylamine; DMF refers to N,N′-dimethylformamide; DMSO refers to dimethyl sulfoxide; EDT refers to 1,2-ethanedithiol (1,2-ethylenedithiol); ESI-MS refers to electrospray ionization mass spectrometry; EtOAc refers to ethyl acetate; Et₂O refers to diethyl ether; Fmoc refers to (9H-fluoren-9-ylmethoxy) carbonyl; HBTU refers to 2-(1H-benzotriazol-1-yl)-1,1,3,3-tetramethyluronium hexafluorophosphate; HOBt refers to N-hydroxybenzotriazole; HPLC refers to high-performance liquid chromatography; OBt refers to O-benzotriazolyl; MeOH refers to methanol; NMR refers to nuclear magnetic resonance; Pbf refers to 2,2,4,6,7-pentamethyl-dihydrobenzofuran-5-sulfonyl; RP-HPLC refers to reverse-phase high-performance liquid chromatography; SPE refers to solid-phase extraction; SPPS refers to solid-phase peptide synthesis; Bu refers to tert-butyl; THF refers to tetrahydrofuran; TFA refers to trifluoroacetic acid; TIPS refers to triisopropylsilane; TMS refers to tetramethylsilane; and TRT refers to trityl.

### Synthesis of CC16 Compound Analogs:

Chemical Materials: N-α-Fmoc-protected amino acids, HBTU, DIC, Oxima, and HOBt were purchased from P3 Biosystem (Louisville, KY) or from Novabiochem (San Diego, CA). Rink and Wang resins were acquired from Rapp Polymere (Tubingen, Germany) or from Novabiochem (San Diego, CA). For the Nα-Fmoc-protected amino acids, the following side chain protecting groups were used: Fmoc-Arg (N^{w}-Pbf), Fmoc-Asp (OtBu), Fmoc-Asp(OAll), Fmoc-Gln (Nc-Trt), Fmoc-Glu (OtBu), Fmoc-Glu(OAll), Fmoc-Lys (Ne-Boc), Fmoc-Lys (Ne-Aloc), Fmoc-Thr (tBu). Reagent-grade solvents, reagents, and acetonitrile for HPLC were acquired from VWR (West Chester, PA) or Aldrich-Sigma (Milwaukee, WI) and were used without further purification unless otherwise noted. Chemicals and reagents were obtained from Sigma-Aldrich or TCI. The solid-phase synthesis was performed in fritted syringes using a Domino manual synthesizer obtained from Torviq (Tucson, AZ).

General Synthesis: All solution phase reactions were conducted under an Argon (Ar) atmosphere using oven-dried glassware. All chemicals were obtained from commercial sources and used without further purification. ¹H NMR spectra were recorded on a Bruker-DRX-300 MHz instrument with chemical shifts reported relative to TMS (0.0 ppm) and residual DMSO (2.50 ppm). Proton-decoupled ¹³C NMR spectra were referenced to CDCl₃ (77.0 ppm) as well as DMSO (39.51 ppm). Low-resolution mass spectra were obtained on AGILENT (HP) MDS 1100 using AP-ESI. High-resolution mass spectra (HRMS) were recorded on a JEOL HX110A instrument.

General Solid-phase Peptide Synthesis: Peptides were prepared as previously published by solid-phase synthesis as summarized in FIG. 1, on Rink Amide Polystyrene (PS) resin (0.68 mmol/g) or on Wang PS resin (0.54 mmol/g) using a Fmoc//Bu synthetic strategy and standard HOBt/DIC, HBTU, or symmetric anhydride activations. N^{α}-Fmoc amino acid was double coupled using preactivated 0.3 M HOBt esters and HBTU/2,4,6-collidine or symmetric anhydride couplings.

The Rink resin was washed with DMF, and the N^{α}-Fmoc protecting group was removed with 1:10 piperidine in DMF (1 I 2 min and 1 I 20 min). The resin was washed successively with DMF, DCM, DMF, a solution of 0.05 mM solution of Bromophenol Blue in 0.2 M HOBt in DMF, then DMF. The N^{α}-Fmoc amino acids were coupled using pre-activated 0.3 M HOBt esters in DMF-DCM mixture (3 equiv of acid, 3 equiv of HOBt, and 3 equiv of DIC). The resin slurry was stirred for 2 h or until the bromophenol test became negative. If the test failed, the resin was washed with DMF, and the amino acid was coupled again by the HBTU/2,4,6-lutidine procedure (0.3 M solution of 3 equiv of acid, 3 equiv of HBTU, and 6 equiv of 2,4,6-lutidine in DMF) for 3 h or by a preformed symmetric anhydride (3 equiv of acid and 3 equiv of DIC in a 1:1 DMF-THE mixture) until Kaiser test was negative. If the couplings did not result in a negative Kaiser test, the resin was washed with DMF, and the free amino groups were capped with 1.0 M acetic anhydride/pyridine in DMF for 20 minutes. After all coupling sequentially to the Rink amide resin, the resin was capped with 1.0 M acetic anhydride/pyridine in DMF for 20 minutes, washed with DMF, washed with DCM, dried under vacuum, and then stored in the refrigerator. For cleavage of the final product, the resin was washed thoroughly with DMF (3×) and DCM (7×). A cleavage cocktail (10 mL per 1 g of the resin) comprising or consisting of CF₃CO₂H (95%), EDT (3%), and TIPS (2%) was injected into the resin and the mixture was agitated at room temperature for 2-4 h. The solution was filtered, the resin was washed with CF₃CO₂H (2×3 min), the liquid phases were collected and concentrated under a stream of nitrogen, and the product was precipitated using cold Et₂O. The crude product was washed three times with cold Et₂O, lyophilized, purified, and characterized as described above. The pure compounds were dissolved in deionized (DI) or DMSO at approximately 1-5 mM concentrations, and concentration was determined by Trp-HPLC measurement.

General Protocol for Cyclic Peptides: Cyclic peptides were prepared as previously described in the general synthetic protocol as summarized in FIG. 2 (compound C966) utilizing Rink Amide PS resin (0.68 mmol/g). Standard Fmoc-protected amino acids were employed for the synthesis, except the Aloc-protected lysine building block Fmoc-Lys(Aloc)-OH and the O-allyl ester-protected aspartic acid Fmoc-Asp(OAll) or O-allyl ester-protected glutamic acid Fmoc-Glu(OAll) were used for the incorporation of orthogonally protected Lys/Asp or Lys/Glu pair at positions needed for cyclization; Fmoc-Lys(Aloc)-OH or Fmoc-Asp(OAll)-OH or Fmoc-Glu(OAll)-OH were coupled at the positions when appropriate. Usually, 3 equiv of Fmoc-protected amino acid, 3 equiv of HCTU, and 3 equiv of DIPEA were used in coupling cycles. Fmoc deprotection was achieved by treatment with 10% piperidine/DMF for 2+20 min, except after the incorporation of allyl esters was Fmoc deprotection time decreased to 2+7 min. For N-terminal acetylated peptides, the N-terminus was acetylated with 1.0 M acetic anhydride/pyridine in DMF for 20 minutes. For Aloc/Allyl deprotection, the resin was washed with DCM and then flushed with argon for 3-10 minutes. A cleavage mixture 5 equiv of dimethylbarbituric acid, 0.2 eq. Pd(0)[(Ph3P)4] in DCM (0.1 equiv) is flushed with argon and injected. The reaction mixture is stirred for 30 min then repeated. The resin is washed with DCM, 10% DIEA in DCM (3×2 min), DMF (2×), 1% Sodium diethyldithiocarbamate trihydride in DMF (2×20 min), 10% DIEA in DMF (3×2 min), and DMF. For cyclization, a solution of PyBOP (4 equiv, 0.5M) and DIPEA (8 equiv) in DMF was added to the resin. Full conversion to the side chain cross-linked lactam was monitored by cleaving a small sample than HPLC (usually ~6 h). After completion, the resin was washed with DMF and DCM. For cleavage of the final product, a cleavage cocktail (10 mL per 1 g of the resin) consisting of CF₃CO₂H (95%), EDT (3%), and TIPS (2%) was injected into the resin and the mixture was agitated at room temperature for 2-4 h. The crude product was obtained as in general solid-phase procedure.

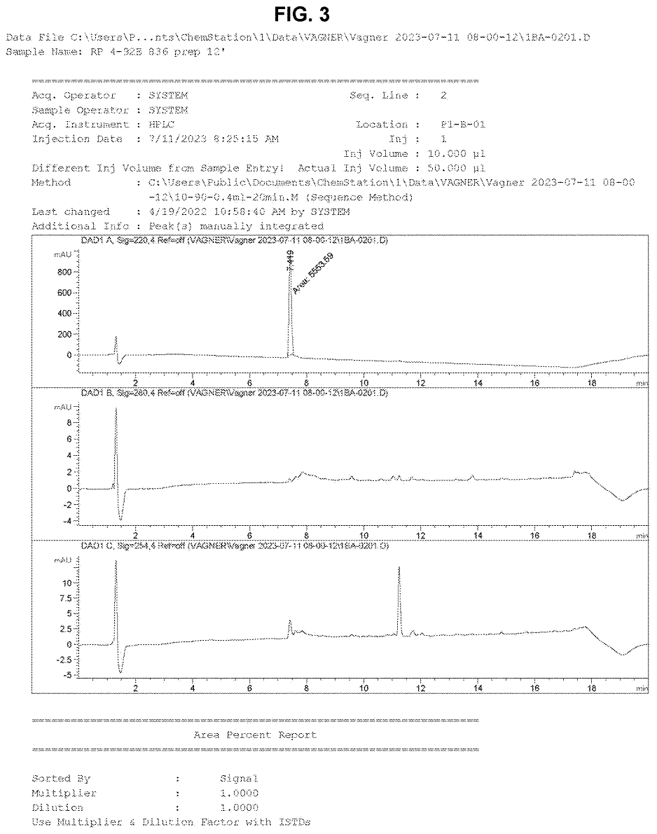

General QC and purification procedure: The purity of products was checked by analytical RP-HPLC using an Agilent 2600 system with a 3.0×100 mm Agilent Poroshell 120 EC-C18 2.7-micron column eluting with a solvent gradient A: B, where solvent A: 0.1% TFA in water and solvent B: 0.09% TFA in acetonitrile:water (9:1), over 15 min at a flow rate of 0.4 mL/min. Purification of ligands was achieved on a Waters 600 HPLC using a reverse phase column (Vydac C18, 15-20 μm, 22×250 mm). Peptides were eluted with a linear gradient of CH₃CN/0.1% CF₃CO₂H at a flow rate of 5.0 mL/min. Separation was monitored at 230 and 280 nm. Size exclusion chromatography was performed on a borosilicate glass column (2.6×250 mm, Sigma, St. Louis, MO) filled with medium-sized Sephadex G-25 or G-10. The compounds were eluted with an isocratic flow of 1.0 M aqueous acetic acid. The pure compounds were dissolved in DI water or DMSO at approximately 1-5 mM concentrations. Accurate concentrations were determined by HPLC at 280 nm. A solution of D-Trp in water or DMSO, accordingly, was co-injected as an internal standard. The product C836 was 56 mg as a white lyophilizate (yield 21%, calc 971.04 found for (M+2H) 2⁺, HPLC >99% see FIG. 3). Yield and retention times are depicted in Tables 1-3.

Table 1: The bolded and underlined amino acids (i.e., Ala) indicate the specific alanine residue that was used in the activity scanning.

TABLE 1
The bolded and underlined amino acids (i.e., Ala) indicate the specific alanine
residue that was used in the activity scanning.

| | | | | | SEQ ID |
|---|---|---|---|---|---|
| Prot | Structures of Ala-scan peptides | MW | Rtª | Yield^{b} | NO: |
| 977 | Ac-Asp-Gln-Asp-Nle-Arg-Glu-Ala-Gly-Ala-Gln- | 1910.02 | 7.49 | 3.9 | 1 |
| | Leu-Lys-Lys-Leu-Val-Asp-Ala-NH₂ | | | | |
| 978 | Ac-Asp-Gln-Asp-Nle-Arg-Glu-Ala-Gly-Ala-Gln- | 1896.04 | 7.34 | 1.0 | 2 |
| | Leu-Lys-Lys-Leu-Val-Ala-Thr-NH₂ | | | | |
| 979 | Ac-Asp-Gln-Asp-Nle-Arg-Glu-Ala-Gly-Ala-Gln- | 1912.00 | 6.91 | 2.9 | 3 |
| | Leu-Lys-Lys-Leu-Ala-Asp-Thr-NH₂ | | | | |
| 980 | Ac-Asp-Gln-Asp-Nle-Arg-Glu-Ala-Gly-Ala-Gln- | 1897.99 | 6.44 | 2.3 | 4 |
| | Leu-Lys-Lys-Ala-Val-Asp-Thr-NH₂ | | | | |
| 981 | Ac-Asp-Gln-Asp-Nle-Arg-Glu-Ala-Gly-Ala-Gln- | 1882.97 | 7.45 | 5.4 | 5 |
| | Leu-Lys-Ala-Leu-Val-Asp-Thr-NH₂ | | | | |
| 987 | Ac-Asp-Gln-Asp-Nle-Arg-Glu-Ala-Gly-Ala-Gln- | 1882.97 | 7.94 | 5.1 | 6 |
| | Leu-Ala-Lys-Leu-Val-Asp-Thr-NH₂ | | | | |
| 988 | Ac-Asp-Gln-Asp-Nle-Arg-Glu-Ala-Gly-Ala-Gln- | 1897.99 | 6.16 | 2.7 | 7 |
| | Ala-Lys-Lys-Leu-Val-Asp-Thr-NH₂ | | | | |
| 989 | Ac-Asp-Gln-Asp-Nle-Arg-Glu-Ala-Gly-Ala-Ala- | 1883.01 | 7.91 | 4.9 | 8 |
| | Leu-Lys-Lys-Leu-Val-Asp-Thr-NH₂ | | | | |
| 990 | Ac-Asp-Gln-Asp-Nle-Arg-Glu-Ala-Ala-Ala-Gln- | 1954.05 | 8.08 | 4.5 | 9 |
| | Leu-Lys-Lys-Leu-Val-Asp-Thr-NH₂ | | | | |
| 991 | Ac-Asp-Gln-Asp-Nle-Arg-Ala-Ala-Gly-Ala-Gln- | 1882.03 | 7.25 | 4.1 | 10 |
| | Leu-Lys-Lys-Leu-Val-Asp-Thr-NH₂ | | | | |
| 992 | Ac-Asp-Gln-Asp-Nle-Ala-Glu-Ala-Gly-Ala-Gln- | 1854.97 | 7.77 | 3.3 | 11 |
| | Leu-Lys-Lys-Leu-Val-Asp-Thr-NH₂ | | | | |
| 993 | Ac-Asp-Gln-Asp-Ala-Arg-Glu-Ala-Gly-Ala-Gln- | 1897.99 | 6.48 | 3.9 | 12 |
| | Leu-Lys-Lys-Leu-Val-Asp-Thr-NH₂ | | | | |
| 994 | Ac-Asp-Gln-Ala-Nle-Arg-Glu-Ala-Gly-Ala-Gln- | 1896.04 | 7.81 | 2.8 | 13 |
| | Leu-Lys-Lys-Leu-Val-Asp-Thr-NH₂ | | | | |
| 995 | Ac-Asp-Ala-Asp-Nle-Arg-Glu-Ala-Gly-Ala-Gln- | 1883.01 | 7.39 | 4.5 | 14 |
| | Leu-Lys-Lys-Leu-Val-Asp-Thr-NH₂ | | | | |
| 996 | Ac-Ala-Gln-Asp-Nle-Arg-Glu-Ala-Gly-Ala-Gln- | 1896.04 | 7.60 | 3.5 | 15 |
| | Leu-Lys-Lys-Leu-Val-Asp-Thr-NH₂ | | | | |

^{a}Pure peptides were analyzed using an Agilent 2600 system with a 3.0 x 100 mm Agilent Poroshell 120 EC-C18 2.7-micron column eluting with a solvent gradient A:B, where solvent A: 0.1% TFA in water and solvent B: 0.09% TFA in acetonitrile:water (9:1), over 15 min at a flow rate of 0.4 mL/min. The specific retention time (Rt) are described. The purity of HPLC-purified compounds exceeded 95%.
^{b}Yield is weight amount (mg) of pure peptide

TABLE 2
The bolded and underlined amino acids indicate which amino acids were changed
in the compound compared to the wild-type CC16 protein.

| | Structures of D-scan peptides | | | SEQ ID |
|---|---|---|---|---|
| Prot | (MW 1940.03 for all structures) | Rtª | Yield^{b} | NO: |
| 982 | Ac-Asp-Gln-Asp-Nle-Arg-Glu-Ala-Gly-Ala-Gln-Leu-Lys- | 7.75 | 4.5 | 16 |
| | Lys-Leu-Val-Asp-thr-NH₂ | | | |
| 983 | Ac-Asp-Gln-Asp-Nle-Arg-Glu-Ala-Gly-Ala-Gln-Leu-Lys- | 7.02 | 2.7 | 17 |
| | Lys-Leu-Val-asp-Thr-NH₂ | | | |
| 984 | Ac-Asp-Gln-Asp-Nle-Arg-Glu-Ala-Gly-Ala-Gln-Leu-Lys- | 6.63 | 3.6 | 18 |
| | Lys-Leu-val-Asp-Thr-NH₂ | | | |
| 985 | Ac-Asp-Gln-Asp-Nle-Arg-Glu-Ala-Gly-Ala-Gln-Leu-Lys- | 6.70 | 2.5 | 19 |
| | Lys-leu-Val-Asp-Thr-NH₂ | | | |
| 986 | Ac-Asp-Gln-Asp-Nle-Arg-Glu-Ala-Gly-Ala-Gln-Leu-Lys- | 6.41 | 2.9 | 20 |
| | lys-Leu-Val-Asp-Thr-NH₂ | | | |
| 997 | Ac-Asp-Gln-Asp-Nle-Arg-Glu-Ala-Gly-Ala-Gln-Leu-lys- | 6.52 | 5.2 | 21 |
| | Lys-Leu-Val-Asp-Thr-NH₂ | | | |
| 998 | Ac-Asp-Gln-Asp-Nle-Arg-Glu-Ala-Gly-Ala-Gln-leu-Lys- | 6.49 | 3.3 | 22 |
| | Lys-Leu-Val-Asp-Thr-NH₂ | | | |
| 999 | Ac-Asp-Gln-Asp-Nle-Arg-Glu-Ala-Gly-Ala-gln-Leu-Lys- | 6.79 | 6.6 | 23 |
| | Lys-Leu-Val-Asp-Thr-NH₂ | | | |
| 1000 | Ac-Asp-Gln-Asp-Nle-Arg-Glu-Ala-Gly-ala-Gln-Leu-Lys- | 6.48 | 3.9 | 24 |
| | Lys-Leu-Val-Asp-Thr-NH₂ | | | |
| 1001 | Ac-Asp-Gln-Asp-Nle-Arg-Glu-ala-Gly-Ala-Gln-Leu-Lys- | 6.72 | 5.7 | 25 |
| | Lys-Leu-Val-Asp-Thr-NH₂ | | | |
| 1002 | Ac-Asp-Gln-Asp-Nle-Arg-glu-Ala-Gly-Ala-Gln-Leu-Lys- | 6.80 | 3.7 | 26 |
| | Lys-Leu-Val-Asp-Thr-NH₂ | | | |
| 1003 | Ac-Asp-Gln-Asp-Nle-arg-Glu-Ala-Gly-Ala-Gln-Leu-Lys- | 6.77 | 3.2 | 27 |
| | Lys-Leu-Val-Asp-Thr-NH₂ | | | |
| 1004 | Ac-Asp-Gln-Asp-nle-Arg-Glu-Ala-Gly-Ala-Gln-Leu-Lys- | 6.79 | 3.4 | 28 |
| | Lys-Leu-Val-Asp-Thr-NH₂ | | | |
| 1005 | Ac-Asp-Gln-asp-Nle-Arg-Glu-Ala-Gly-Ala-Gln-Leu-Lys- | 6.46 | 4.3 | 29 |
| | Lys-Leu-Val-Asp-Thr-NH₂ | | | |
| 1006 | Ac-Asp-gln-Asp-Nle-Arg-Glu-Ala-Gly-Ala-Gln-Leu-Lys- | 7.14 | 3.1 | 30 |
| | Lys-Leu-Val-Asp-Thr-NH₂ | | | |
| 1007 | Ac-asp-Gln-Asp-Nle-Arg-Glu-Ala′. | 7.62 | 4.2 | 31 |
| | -Gly-Ala-Gln-Leu-Lys-Lys-Leu-Val-Asp-Thr-NH₂ | | | |

TABLE 3
The bolded and underlined amino acids indicate which amino acids were changed
in the compound compared to the wild-type CC16 protein. The ″c″ outside the bracketed
amino acids indicates which amino acids were cyclicized in the analog.

| | | | | | SEQ ID |
|---|---|---|---|---|---|
| Prot | Structures truncated and cyclic analogs of C836 | MW | Rtª | Yield^{b} | NO: |
| 969 | Ac-Asp-Gln-Asp-Nle-Arg-Glu-Ala-Gly-Ala-Gln- | 1838.99 | 7.41 | 5.6 | 32 |
| | Leu-Lys-Lys-Leu-Val-Asp-NH₂ | | | | |
| 970 | Ac-Asp-Gln-Asp-Nle-Arg-Glu-Ala-Gly-Ala-Gln- | 1839.97 | 6.98 | 5.7 | 33 |
| | Leu-Lys-Lys-Leu-Val-Asp-OH | | | | |
| 971 | Ac-Asp-Gln-Asp-Nle-Arg-Glu-Ala-Gly-Ala-Gln- | 1723.96 | 7.31 | 2.1 | 34 |
| | Leu-Lys-Lys-Leu-Val-NH₂ | | | | |
| 972 | Ac-Gln-Asp-Nle-Arg-Glu-Ala-Gly-Ala-Gln-Leu- | 1825.01 | 7.26 | 4.6 | 35 |
| | Lys-Lys-Leu-Val-Asp-Thr-NH₂ | | | | |
| 973 | Ac-Asp-Nle-Arg-Glu-Ala-Gly-Ala-Gln-Leu-Lys- | 1696.95 | 7.27 | 2.7 | 36 |
| | Lys-Leu-Val-Asp-Thr-NH₂ | | | | |
| 974 | Ac-Nle-Arg-Glu-Ala-Gly-Ala-Gln-Leu-Lys-Lys- | 1468.84 | 6.05 | 2.9 | 37 |
| | Leu-Val-Asp-Thr-NH₂ | | | | |
| 966 | Ac-Asp-Gln-Asp-Nle-Arg-[Glu-Ala-Gly-Ala- | 1922.06 | 8.29 | 3.2 | 38 |
| | Lys]c-Leu-Lys-Lys-Leu-Val-Asp-Thr-NH₂ | | | | |
| 967 | Ac-Asp-Gln-Asp-Nle-Arg-Glu-Ala-Gly-Ala-Gln- | 1922.02 | 8.66 | 3.5 | 39 |
| | Leu-[Lys-Lys-Leu-Val-Asp]c-Thr-NH₂ | | | | |
| 968 | Ac-Asp-Gln-Asp-Nle-Arg-Glu-Ala-Gly-Ala-Gln- | 1936.04 | 8.54 | 4.2 | 40 |
| | Leu-[Lys-Lys-Leu-Val-Glu]c-Thr-NH₂ | | | | |

### Example 2

The following is a non-limiting example of the present invention. It is to be understood that said example is not intended to limit the present invention in any way. Equivalents or substitutes are within the scope of the present invention.

In the examples provided below, human recombinant CC16 (rCC16) was delivered either intravenously (IV) via retro-orbital injection or directly to the lungs through oropharyngeal delivery. Both delivery methods are effective in providing protection against airway hyperresponsiveness and respiratory infections. However, only intravenous delivery has been shown to reduce leukocyte recruitment into the lungs.

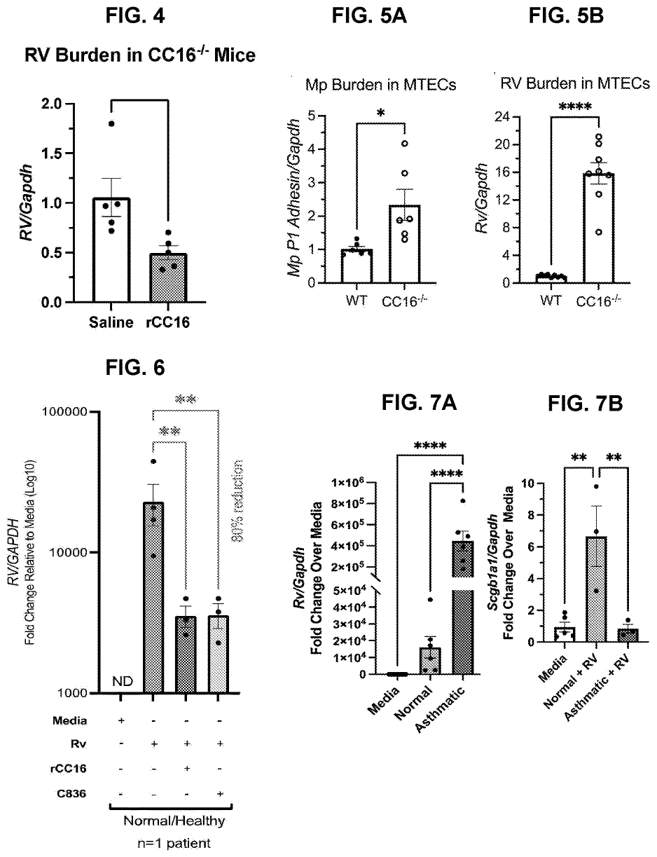

rCC16 treatment decreases human Rhinovirus (RV; RVIB) burden in CC16^{-/-} mice: For these studies, female mice were infected with RV (4×10⁷ plaque-forming unit (PFU)/50 μl) via intranasal instillation for 48 hrs, and mice were either given vehicle (saline) or rCC16 (human recombinant CC16) at the time of infection. RV burden was determined by RT-PCR, and mice receiving rCC16 showed a significant reduction in RV compared to vehicle-treated mice (FIG. 4). This suggests that host responses to the virus can be restored when CC16 is given back to the lungs.

Epithelial cells lacking CC16 have heightened infectivity. CC16^{-/-} mouse tracheal epithelial cells (MTECs) have enhanced Mycoplasma pneumoniae (Mp) infection compared to WT cells (FIG. 5A). In order to determine if the role of CC16 during viral infection was epithelial-driven, as opposed to impacting local immune cells, MTEC cultures were grown from WT and CC16^{-/-} mice at an air-liquid interface (ALI) and challenged with RVIB (Human Rhinovirus 1B). For these studies, MTECs are infected for 4 hrs, after which any non-adherent virus is washed from the transwells. Then, cells are allowed to incubate at 35° C. for 48 hrs, after which RT-PCR is performed for RV infectivity. As shown in FIG. 5B, cells lacking CC16 had significantly enhanced infection with RV after 48 hrs compared to WT cells, suggesting an impairment in anti-viral host responses as well as the impairment in anti-microbial responses observed with Mp (FIG. 5A).

rCC16 and CC16-derived peptidomimetics reduce RV infection in human nasal cells. In order to test if rCC16 added exogenously could protect against RV viral infection, primary human nasal epithelial cells (HNECs) were grown from non-asthma participants at an air-liquid interface (ALI) for 21 days. On the day of infection, RV was added to the apical surface and allowed to incubate for 4 hrs, after which cells were gently washed with media, and either media alone, rCC16 (25 g/ml), or CC16-derived peptidomimetic, C836 (25 g/ml), were added and cells allowed to incubate for an additional 48 hrs. After harvest, RV infectivity was determined by PCR for RV. Both rCC16 and C836 significantly reduced RV infection in human nasal cells, as shown in FIG. 6, by approximately 80%.

Nasal cells from asthma patients have decreased Scgb1a1 (CC16) gene expression and are more susceptible to RV infection. Airway cells isolated from asthma patients are more susceptible to RV infection (FIG. 7A). Thus, the enhanced susceptibility of RV in asthma patient cells may be due to decreased CC16 (also shown in FIG. 7B) and thereby decreased CC16-dependent epithelial antiviral response proteins.

CC16-derived peptidomimetic, C836, given to circulation, is protective in house dust mite (HDM) of allergic airway disease. Upregulation of CC16 by several methods is protective during respiratory challenges, including adenoviral overexpression, vitamin A-induced upregulation, direct delivery of human recombinant CC16 (rCC16), and vesicle-encapsulated nanoparticles. While these strategies to increase CC16 levels in the respiratory space were successful, their translation to the clinic is wrought with various complications, including delivery to patients. For example, adenoviral drug delivery has generally been unsuccessful due to limited efficacy and safety concerns. Similarly, using Vitamin A as a therapeutic is problematic, as excessive doses can lead to severe side effects, including an increased risk of cancer. Nanoparticle-based approaches, while innovative, remain in their infancy, with methods for their formulation and delivery still poorly defined in the literature, posing additional hurdles to their practical application.

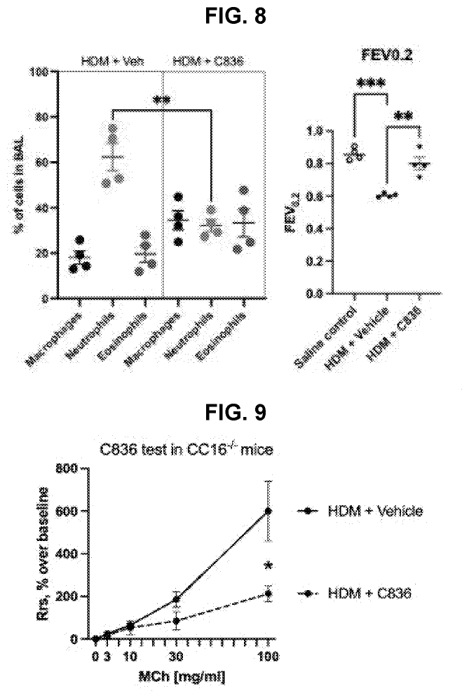

Thus, a small peptidomimetic was designed based on the newly discovered LVD (leucine-valine-aspartic acid) integrin binding domain contained within the CC16 protein. The lead compound, C836, is a 17-mer containing the LVD integrin binding domain. Preliminary data in FIG. 8 (left) shows that when C836 is given to the circulation via intravenous injection (IV), fewer neutrophils are recruited into the lung during HDM challenge. Neutrophils, when present in the lungs of individuals with asthma, are typically associated with more severe and difficult to treat forms of the condition. In COPD, neutrophils are often highly destructive, releasing factors and enzymes that degrade the basement membrane and damage the delicate airway epithelium. Therefore, strategies to reduce neutrophil accumulation or activity in both asthma and COPD would provide significant benefits for overall lung health, potentially improving disease outcomes and reducing airway damage.

Additionally, lung function tests, without provocation, demonstrate that C836 given to HDM-treated mice is protective from loss of lung function as measured by FEV0.2 by invasive PFT tests FIG. 8 (right). Lung function tests are typically performed using methacholine provocation, which induces bronchoconstriction. However, the tests referenced here were conducted without provocation, meaning that when C836 was administered alongside an HDM challenge, it prevented the loss of lung function typically associated with HDM exposure. These tests were conducted at baseline, without the use of a bronchoconstrictor, similar to an asthma patient living a normal day without an attack.

C836 reduces AHR in Cc16^{-/-} mice exposed to HDM. The activity of C836 was tested for its ability to provide protection in the allergic airway model induced by exposure to HDM. For this, CC16^{-/-} mice were used, and the C836 (or vehicle control) was delivered to the airway via the oropharyngeal method approximately 1 hr after each HDM dose on days 0, 7, and 14. Mice were assessed 24 hrs after the last HDM challenge by flexi Vent® and methacholine challenge. As shown in FIG. 9, C836 resulted in significantly lower airway hyperresponsiveness (AHR) compared to HDM-treated given vehicles.

CC16-derived peptidomimetics provide protection against human Rhinovirus infection in airway epithelial cells. Preliminary studies were done in primary nasal cells grown at an ALI. Transwell cells were challenged with RV (moiety of infection (MOI) 0.1) for 4 hrs, after which external RV was washed from the cell surface and test compounds added; uninfected baseline controls will be treated the same by pipetting media. Cells are allowed to incubate for an additional 48 hrs. Three readouts were used for analysis in this screen: RV infectivity, INF-lambda induction, and TNF-alpha induction. An optimal compound for this screening will have a reduction in RV infection, maintain high levels of INF-lambda, and have low levels of TNF-alpha.

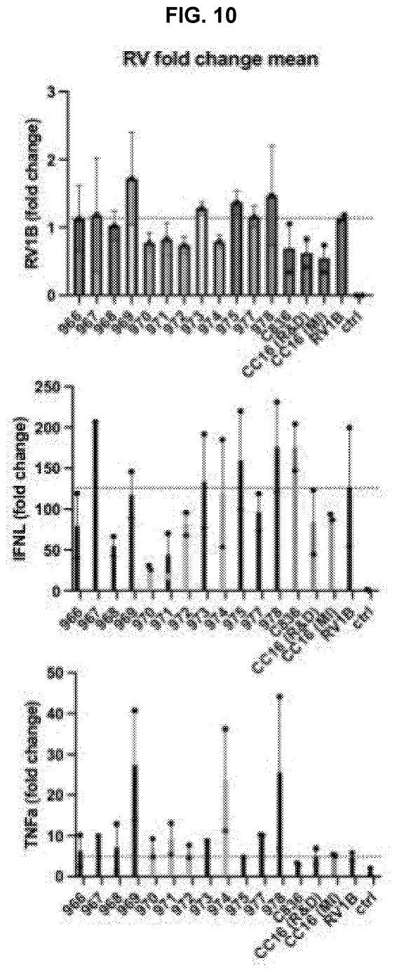

Test compounds are performed in triplicate at an initial dose of 2.5 μM, which has been found to be optimal for rCC16 to inhibit 50% of infection. As shown in FIG. 10, an example of the RV screen shows standard controls [rCC16 from R&D and rCC16 made at UA (MJ)] and active parent compound C836 have activity in reducing RV infectivity. Those compounds that had RV inhibition activity are shown in green (upper panel): 970, 971, 972, and 974. In the middle panel, of those, compounds 972 and 974 maintain INF-lambda levels. However, compound 974 has a spike in TNF-alpha, bottom panel. From this screen for the 3 factors of interest, compound 972 is the best candidate to move forward. Compounds in gray were deemed inactive in this assay. RV burden, INF-lambda, and TNF-alpha are assessed by RT-PCR. Those compounds moving forward will be tested in a dose-response to determine optimal concentrations needed for IC₅₀.

### Example 3

The following is a non-limiting example of the present invention. It is to be understood that said example is not intended to limit the present invention in any way. Equivalents or substitutes are within the scope of the present invention.

Synthesis of 1019 and its analogs: On Chloro-2-Chlorotrityl PS resin (1.3 mmol/g) or on Wang PS resin (0.70 mmol/g) using a Fmoc//Bu synthetic strategy and standard HOBt/DIC, HBTU, or symmetric anhydride activations.¹ N^{a}-Fmoc amino acid was double coupled using preactivated 0.3 M HOBt esters and HBTU/2,4,6-collidine or symmetric anhydride couplings.

The Chloro-2-Chlorotrityl PS resin was washed with DCM. The resin was treated overnight with 0.5M DCM solution of N^{a}-Fmoc-Dap(Aloc) (3 equiv) and DIEA (4 equiv). The resin was washed successively with DCM, and DMF. A Fmoc protecting group was removed with 1:10 piperidine in DMF (1×2 min and 1×20 min). The resin was washed successively with DMF, DCM, DMF, a solution of 0.05 mM solution of Bromophenol Blue in 0.2 M HOBt in DMF, then DMF. The N^{a}-Fmoc amino acid were coupled using pre-activated 0.3 M HOBt esters in DMF-DCM mixture (3 equiv of acid, 3 equiv of HOBt and 3 equiv of DIC). The resin slurry was stirred for 2 h or until the bromophenol test became negative. If the test failed, the resin was washed with DMF and the amino acid was coupled again by the HBTU/2,4,6-lutidine procedure (0.3 M solution of 3 equiv of acid, 3 equiv of HBTU, and 6 equiv of 2,4,6-lutidine in DMF) for 3 h or by a preformed symmetric anhydride (3 equiv of acid and 3 equiv of DIC in a 1:1 DMF-THF mixture) until Kaiser test was negative. If the couplings did not result in a negative Kaiser test, the resin was washed with DMF, and the free amino groups were capped with 1.0M acetic anhydride/pyridine in DMF for 20 minutes. After all coupling sequentially to the resin, the N-terminal of the peptide chain was capped with 2.0M acetic anhydride/pyridine in DCM for 20 minutes or coupled with proprietary building block, washed with DMF and DCM.

The side chain of Dap was elongated starting with deprotection of the Aloc group. The resin was treated with a solution of catalytic amount Palladium (0) Tetrakistriphenylphosphine Pd(Ph3P)4, 0.5M N,N′-dimethylbarbituric acid in DCM for 30 minutes. The cleaved resin was washed with DCM and 1.0M DIEA in DMF. A Fmoc protected Leucine coupling and deprotection was carried out as described in above procedure. The resin was treated with 0.5M solution of 2-Methylphenylisocyanate in DMF (overnight) to form urea. After final coupling, the resin was washed with DMF and DCM.

General QC and purification procedures were carried out as described in peptide protocol.

C-terminal modifications: In compound 1019 Z equals OH as simple carboxylic acid C-terminal end. However, in series 1009-1016, Z is a residue aspartic acid (compounds 1021 and 1022) of dipeptide LD (compound 1009), dipeptide VD (compound 1010), dipeptide DG (compound 1014), dipeptide NleG (compound 1015) or dG (compound 1016); a residue of tetrapeptide LDVP (compound 1011). Optionally, in some embodiments, isosteric structures mimicking C-terminal carboxylate —COOH, including but not limited to hydroxamic acid-CONHOH, tetrazole, or sulfonamide-CONHSO2R may be used to modify the C-terminal.

Acylated diaminopropionic acid Dap: Only two compounds contain acylated Dap (compound 1019 and 1020). The rest of modifications contain either 2-(4-(3-(o-tolyl) ureido)phenyl) acetic acid coupled to side chain of Dap via leucine residue (compounds 1017, 1018, 1021 and 1022) or the residue of Dap was substituted with 2-methyl phenyl urea analog of 4-aminophenylalanine (compounds 1009-1016). This position offers two sides for modification: instead of Leu residue, use other lipophilic amino acids like Val, Phe, Cyclohexyl alanine, homoLeu etc. The other modification uses substitution of phenyl ring either with alkyl branched alkyl, or substitutions on the ring.

Peptide residues A1, A6, A7, A8, and A9: Data from Ala and d-scan showed that these positions are important for activity likely due to stabilization of helical conformation. We propose to claim substitutions of natural sequence with simple helix-promoting residues such as: Ala, Aib, and Cyclohexylglycine.

X (N-terminus): In series 1028-1031, modifications were made to the acetyl group of 1019.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A composition comprising a CC16-derived analog.

2. The composition of claim 1, wherein the CC16-derived analog comprises compound C836 according to the sequence: SEQ ID NO: 41.

3. The composition of claim 1, wherein the CC16-derived analog comprises a sequence according to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

4. The composition of claim 1, wherein the CC16-derived analog is according to the formula: 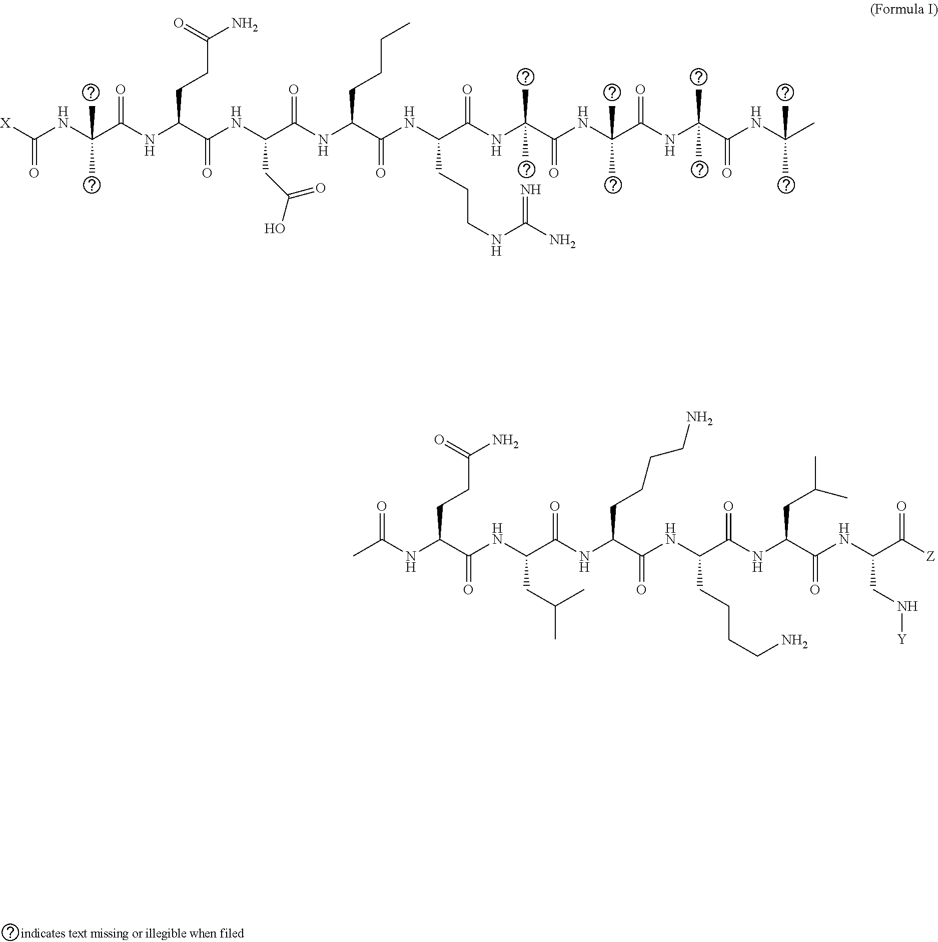
wherein X is an alkyl, a branched alkyl, an acetyl, an isopropyl, or an isobutyl; Z is OH, NH2, NH-alkyl, dipeptide, or an amino acid residue; A is an H, methyl, cyclohexyl, isopropyl, isobutyl, butyl, or an amino acid residue selected from the amino acid group consisting of glutamic acid, aspartic acid, alanine, cyclohexyl glycine, cyclohexyl alanine, amino-isobutyric acid and Y is 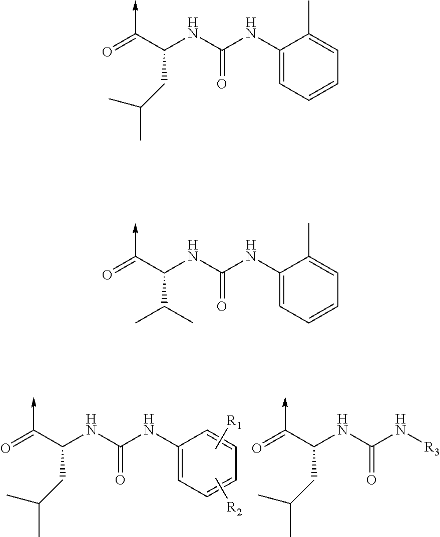
wherein R1, R2 is an H, methyl, a halogen, or OMe, and wherein R3 is a linear or branched alkyl.

5. The composition of claim 4, wherein Z is an amino acid residue selected from the group consisting of proline, leucine, valine, and aspartic acid.

6. The composition of claim 4, wherein Z is a mimic of carboxylic acid selected from the group consisting of hydroxamic acid, urea, N-alkylated urea, N-phenylurea, 2-methyl-N-phenylurea.

7. The composition of claim 4, wherein Y is a residue of aminoacid selected from group of glycine, alanine valine, leucine, isoleucine, phenylalanine, elongated in N-termini with phenylurea, 2-methylphenylurea, 4-bromophenylurea; sulfonylated or acylated

8. The composition of claim 4, wherein A is an amino acid residue selected from the group consisting of alanine or D-alanine.

9. The composition of claim 4, wherein R3 is isobutyl or isovaleryl.

10. The composition of claim 4, wherein the CC16-derived analog is according to the formula:

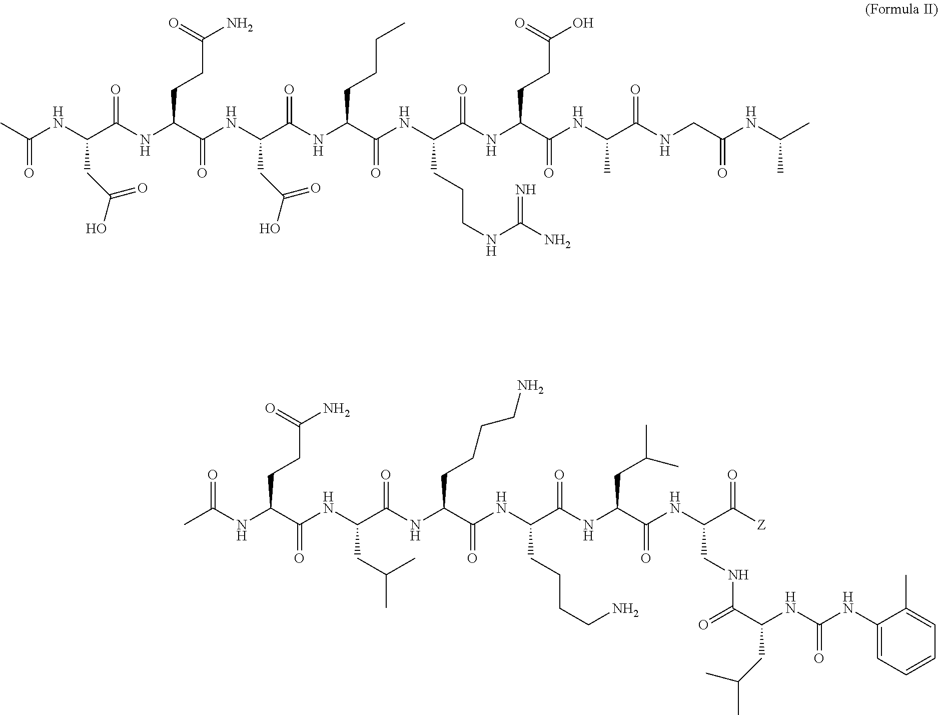

11. The composition of claim 1, wherein the CC16-derived analog is selected from the group consisting of:

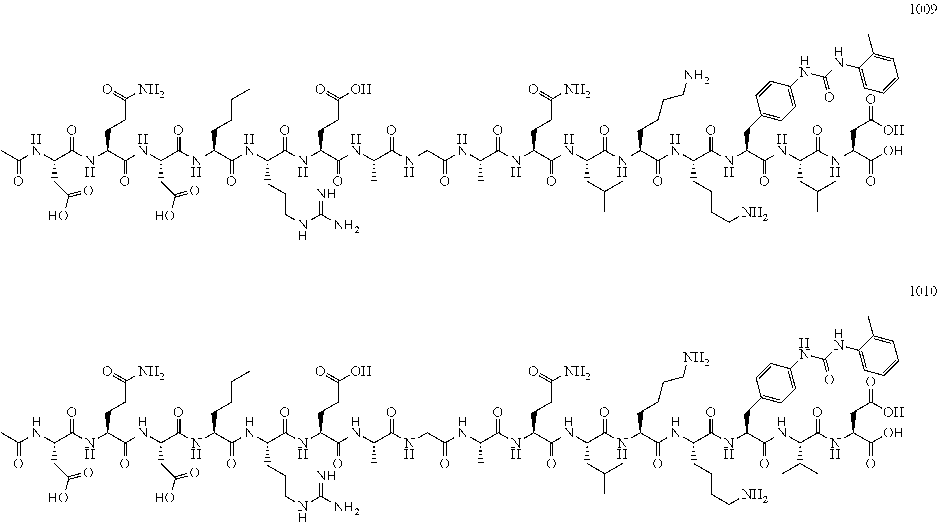

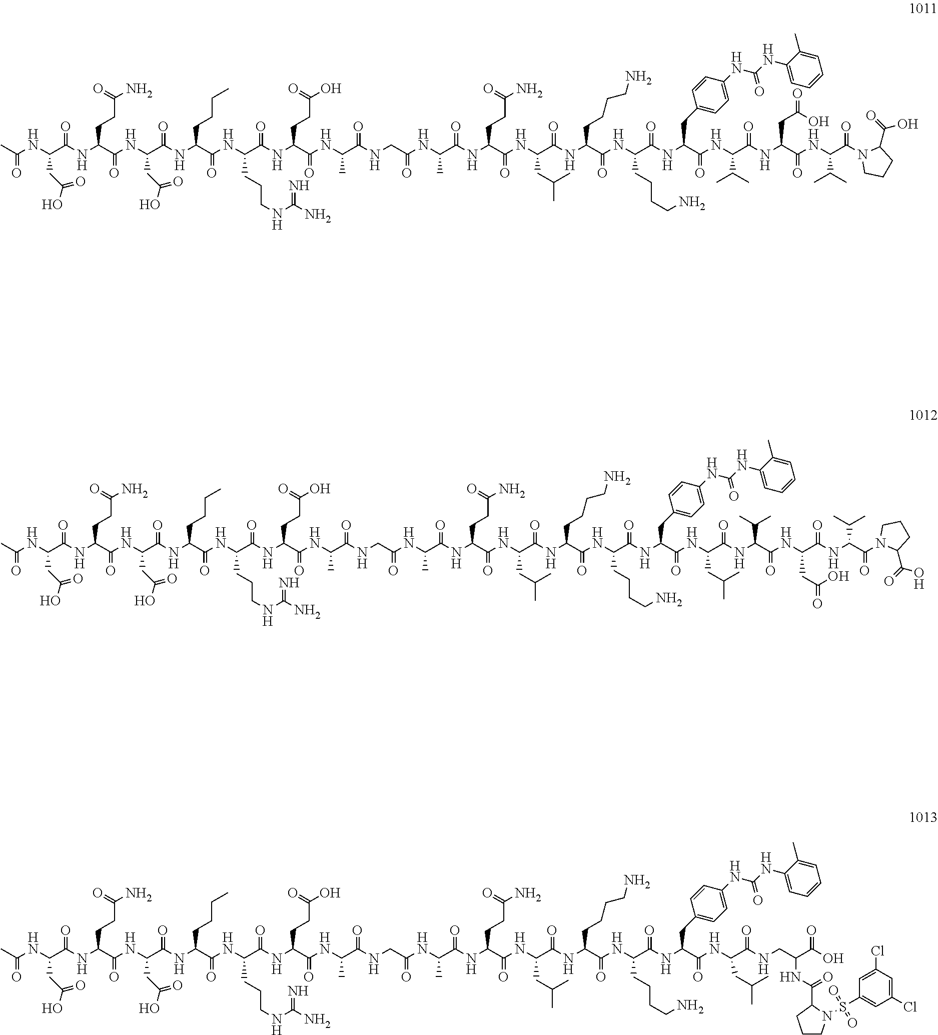

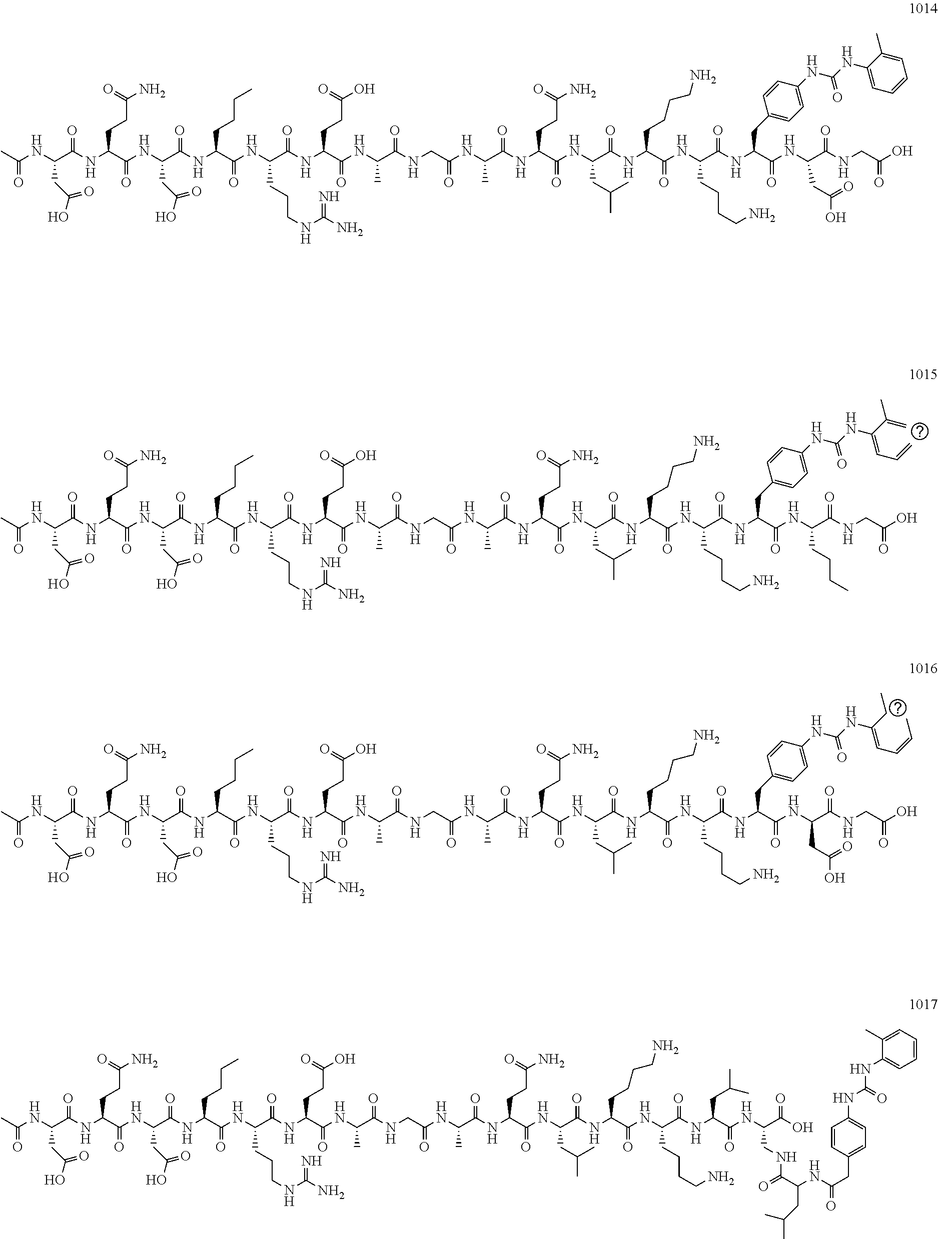

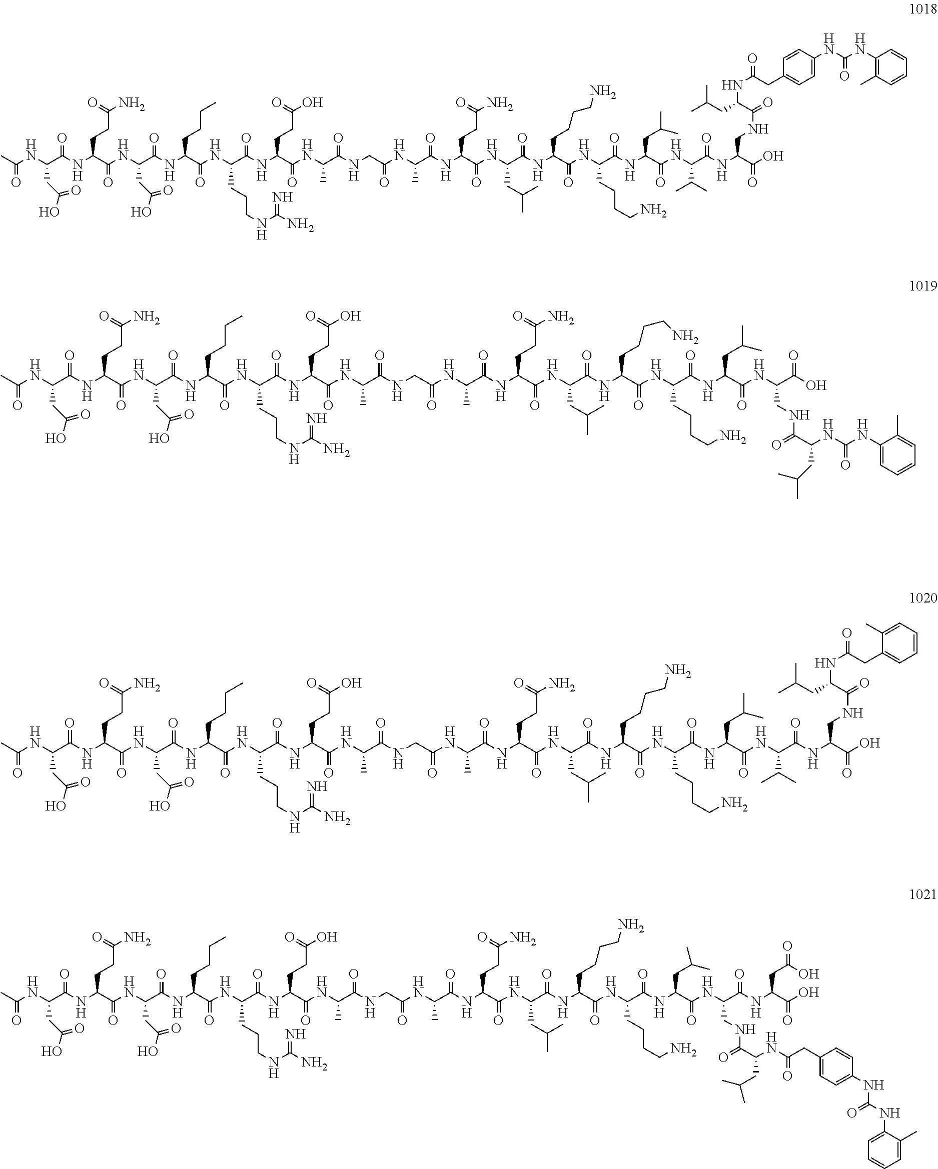

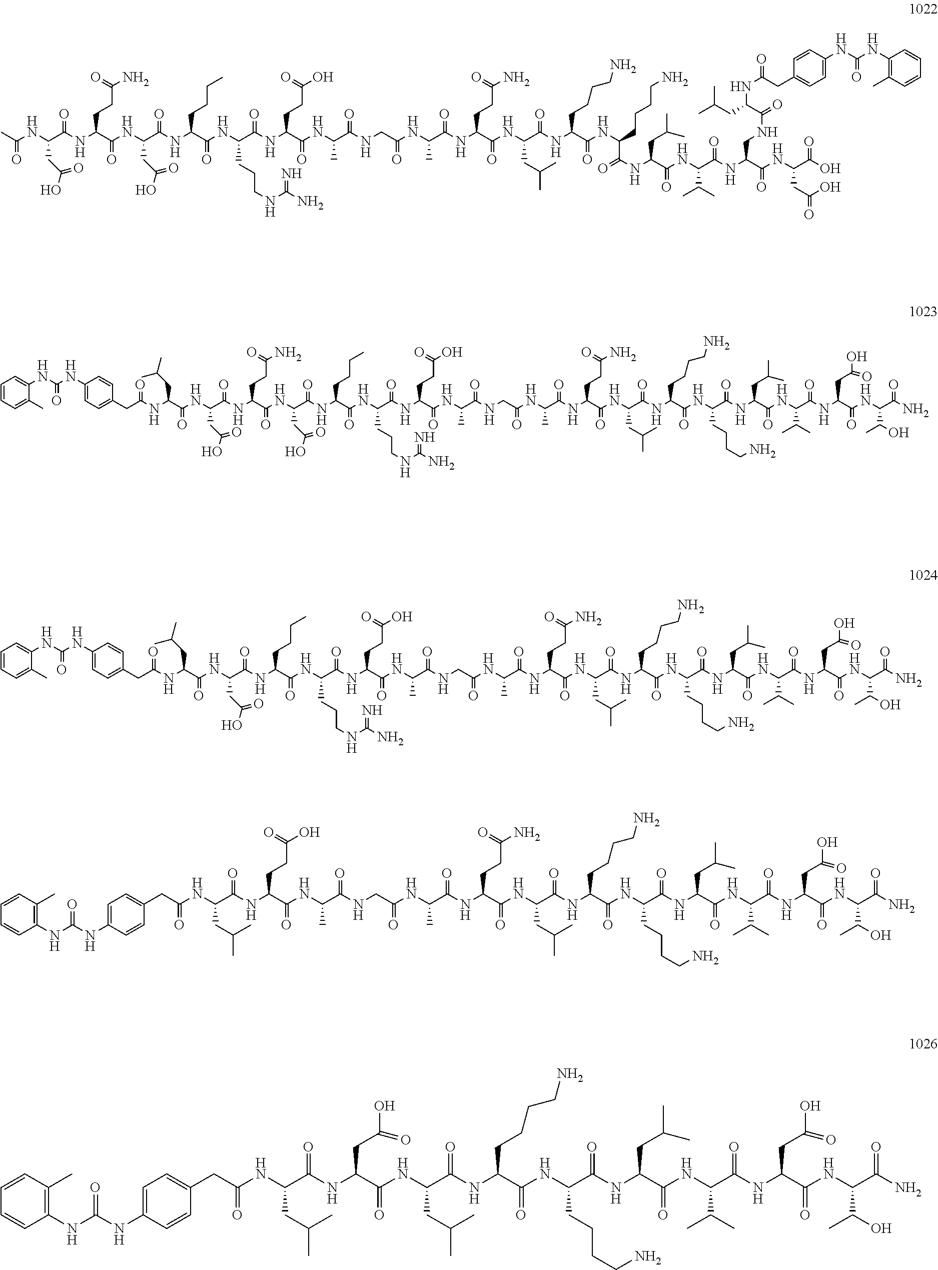

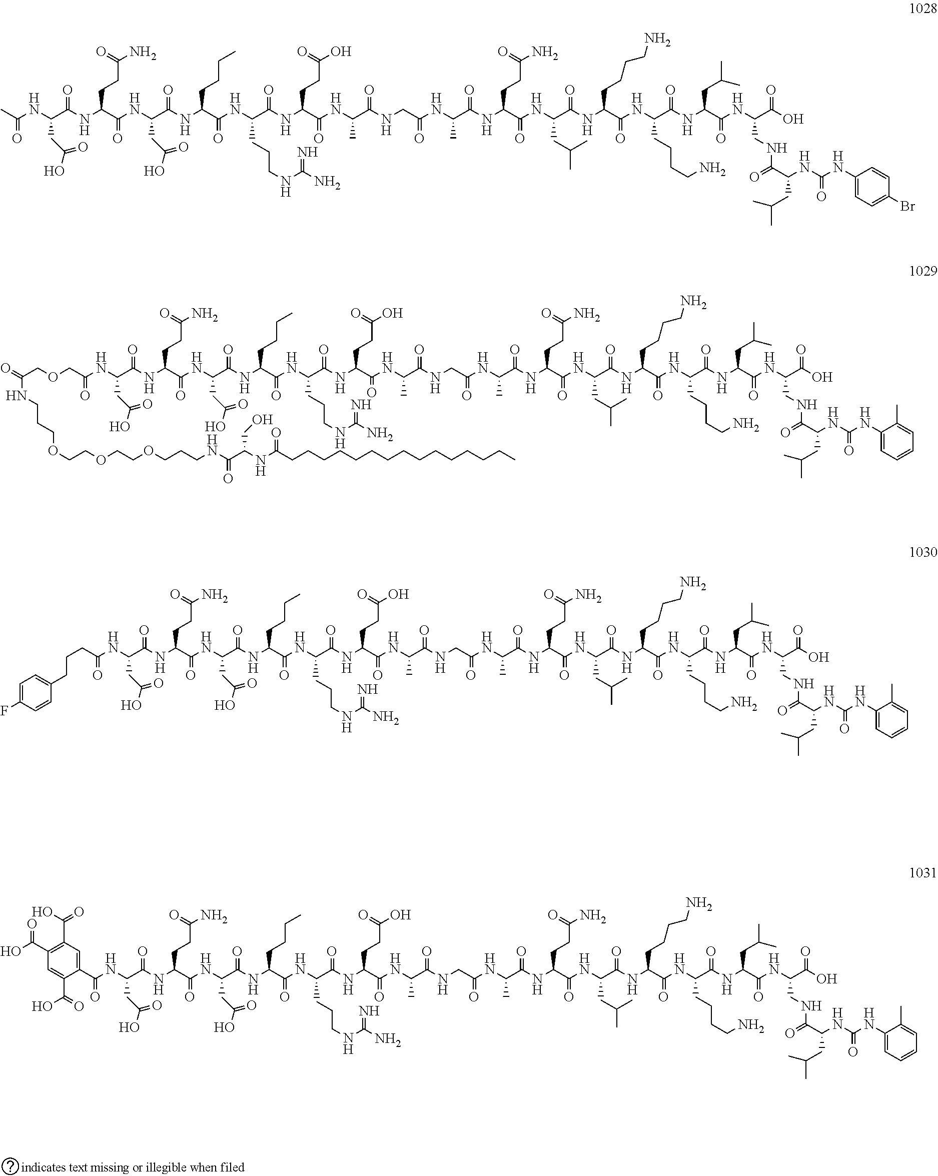

12. The composition of claim 1, wherein the CC16-derived analog further comprises an N-terminal acetylation.

13. The composition of claim 1, wherein the CC16-derived analog further comprises a C-terminal amidation.

14. The composition of claim 1, wherein the CC16-derived analog is helical.

15. The composition of claim 1, wherein at least a portion of the CC16-derived analog is cyclic.

16. The composition of claim 1, wherein the CC16-deri [symbol] a full-length CC16 protein.

17. The composition of claim 1, wherein the composition is used to treat inflammatory, neoplastic, and obstructive lung diseases.

18. The composition of claim 1, wherein the composition is in a preparation for aerosolization, subcutaneous injection, or pulmonary delivery.

19. The composition of claim 1, wherein the composition is administered via nasal inhalation, subcutaneously, or orally.

20. A method of treating inflammatory, neoplastic, or obstructive lung diseases in a subject in need thereof, the method comprising administering an therapeutically effective amount of a composition according to claim 1 to the subject.


SEQUENCE LISTING (41 sequences)

SEQ ID NO: 1 - protein, 17 aa, synthetic construct
  MOD_RES 4: note = Nle
DQDXREAGAQLKKLVDA

SEQ ID NO: 2 - protein, 17 aa, synthetic construct
  MOD_RES 4: note = Nle
DQDXREAGAQLKKLVAT

SEQ ID NO: 3 - protein, 17 aa, synthetic construct
  MOD_RES 4: note = Nle
DQDXREAGAQLKKLADT

SEQ ID NO: 4 - protein, 17 aa, synthetic construct
  MOD_RES 4: note = Nle
DQDXREAGAQLKKAVDT

SEQ ID NO: 5 - protein, 17 aa, synthetic construct
  MOD_RES 4: note = Nle
DQDXREAGAQLKALVDT

SEQ ID NO: 6 - protein, 17 aa, synthetic construct
  MOD_RES 4: note = Nle
DQDXREAGAQLAKLVDT

SEQ ID NO: 7 - protein, 17 aa, synthetic construct
  MOD_RES 4: note = Nle
DQDXREAGAQAKKLVDT

SEQ ID NO: 8 - protein, 17 aa, synthetic construct
  MOD_RES 4: note = Nle
DQDXREAGAALKKLVDT

SEQ ID NO: 9 - protein, 17 aa, synthetic construct
  MOD_RES 4: note = Nle
DQDXREAAAQLKKLVDT

SEQ ID NO: 10 - protein, 17 aa, synthetic construct
  MOD_RES 4: note = Nle
DQDXRAAGAQLKKLVDT

SEQ ID NO: 11 - protein, 17 aa, synthetic construct
  MOD_RES 4: note = Nle
DQDXAEAGAQLKKLVDT

SEQ ID NO: 12 - protein, 17 aa, synthetic construct
DQDAREAGAQLKKLVDT

SEQ ID NO: 13 - protein, 17 aa, synthetic construct
  MOD_RES 4: note = Nle
DQAXREAGAQLKKLVDT

SEQ ID NO: 14 - protein, 17 aa, synthetic construct
  MOD_RES 4: note = Nle
DADXREAGAQLKKLVDT

SEQ ID NO: 15 - protein, 17 aa, synthetic construct
  MOD_RES 4: note = Nle
AQDXREAGAQLKKLVDT

SEQ ID NO: 16 - protein, 17 aa, synthetic construct
  MOD_RES 4: note = Nle
  MOD_RES 17: note = D-amino acid
DQDXREAGAQLKKLVDT

SEQ ID NO: 17 - protein, 17 aa, synthetic construct
  MOD_RES 4: note = Nle
  MOD_RES 16: note = D amino acid
DQDXREAGAQLKKLVDT

SEQ ID NO: 18 - protein, 17 aa, synthetic construct
  MOD_RES 4: note = Nle
  MOD_RES 15: note = D amino acid
DQDXREAGAQLKKLVDT

SEQ ID NO: 19 - protein, 17 aa, synthetic construct
  MOD_RES 4: note = Nle
  MOD_RES 14: note = D amino acid
DQDXREAGAQLKKLVDT

SEQ ID NO: 20 - protein, 17 aa, synthetic construct
  MOD_RES 4: note = Nle
  MOD_RES 13: note = D amino acid
DQDXREAGAQLKKLVDT

SEQ ID NO: 21 - protein, 17 aa, synthetic construct
  MOD_RES 4: note = Nle
  MOD_RES 12: note = D amino acid
DQDXREAGAQLKKLVDT

SEQ ID NO: 22 - protein, 17 aa, synthetic construct
  MOD_RES 4: note = Nle
  MOD_RES 11: note = D amino acid
DQDXREAGAQLKKLVDT

SEQ ID NO: 23 - protein, 17 aa, synthetic construct
  MOD_RES 4: note = Nle
  MOD_RES 10: note = D amino acid
DQDXREAGAQLKKLVDT

SEQ ID NO: 24 - protein, 17 aa, synthetic construct
  MOD_RES 4: note = Nle
  MOD_RES 9: note = D amino acid
DQDXREAGAQLKKLVDT

SEQ ID NO: 25 - protein, 17 aa, synthetic construct
  MOD_RES 4: note = Nle
  MOD_RES 7: note = D amino acid
DQDXREAGAQLKKLVDT

SEQ ID NO: 26 - protein, 17 aa, synthetic construct
  MOD_RES 4: note = Nle
  MOD_RES 6: note = D amino acid
DQDXREAGAQLKKLVDT

SEQ ID NO: 27 - protein, 17 aa, synthetic construct
  MOD_RES 4: note = Nle
  MOD_RES 5: note = D amino acid
DQDXREAGAQLKKLVDT

SEQ ID NO: 28 - protein, 17 aa, synthetic construct
  MOD_RES 4: note = Nle
  MOD_RES 4: note = D amino acid
DQDXREAGAQLKKLVDT

SEQ ID NO: 29 - protein, 17 aa, synthetic construct
  MOD_RES 4: note = Nle
  MOD_RES 3: note = D amino acid
DQDXREAGAQLKKLVDT

SEQ ID NO: 30 - protein, 17 aa, synthetic construct
  MOD_RES 4: note = Nle
  MOD_RES 2: note = D amino acid
DQDXREAGAQLKKLVDT

SEQ ID NO: 31 - protein, 17 aa, synthetic construct
  MOD_RES 4: note = Nle
  MOD_RES 1: note = D amino acid
DQDXREAGAQLKKLVDT

SEQ ID NO: 32 - protein, 16 aa, synthetic construct
  MOD_RES 4: note = Nle
DQDXREAGAQLKKLVD

SEQ ID NO: 33 - protein, 16 aa, synthetic construct
  MOD_RES 4: note = Nle
  MOD_RES 16: note = Hydroxyl Group
DQDXREAGAQLKKLVD

SEQ ID NO: 34 - protein, 15 aa, synthetic construct
  MOD_RES 4: note = Nle
DQDXREAGAQLKKLV

SEQ ID NO: 35 - protein, 16 aa, synthetic construct
  MOD_RES 3: note = Nle
QDXREAGAQLKKLVDT

SEQ ID NO: 36 - protein, 15 aa, synthetic construct
  MOD_RES 2: note = Nle
DXREAGAQLKKLVDT

SEQ ID NO: 37 - protein, 14 aa, synthetic construct
  MOD_RES 1: note = Nle
XREAGAQLKKLVDT

SEQ ID NO: 38 - protein, 17 aa, synthetic construct
  MOD_RES 4: note = Nle
  SITE 6..10: note = Glutamic Acid linked to Lysine
DQDXREAGAKLKKLVDT

SEQ ID NO: 39 - protein, 17 aa, synthetic construct
  MOD_RES 4: note = Nle
  SITE 12..16: note = Lysine linked to Aspartic Acid
DQDXREAGAQLKKLVDT

SEQ ID NO: 40 - protein, 17 aa, synthetic construct
  MOD_RES 4: note = Nle
  SITE 12..16: note = Lysine linked to Glutamic Acid
DQDXREAGAQLKKLVET

SEQ ID NO: 41 - protein, 17 aa, synthetic construct
  MOD_RES 4: note = Nle
DQDXREAGAQLKKLVDT